SEQUENCE LISTING (8 sequences)

SEQ ID NO: 1 - DNA, 20 bp, synthetic construct
  misc_feature 1..20: note = PCR primer
  source 1..20: mol_type = other DNA
tcaacagcgacacccactcc

SEQ ID NO: 2 - DNA, 21 bp, synthetic construct
  misc_feature 1..21: note = PCR primer
  source 1..21: mol_type = other DNA
gcccagagccttcagtgtacc

SEQ ID NO: 3 - DNA, 22 bp, synthetic construct
  misc_feature 1..22: note = PCR primer
  source 1..22: mol_type = other DNA
cttgagggcacctacctctgtc

SEQ ID NO: 4 - DNA, 23 bp, synthetic construct
  misc_feature 1..23: note = PCR primer
  source 1..23: mol_type = other DNA
acactttatgtcaatgttgcccc

SEQ ID NO: 5 - DNA, 20 bp, synthetic construct
  misc_feature 1..20: note = PCR primer
  source 1..20: mol_type = other DNA
tgaggtccaccaccctgttg

SEQ ID NO: 6 - DNA, 20 bp, synthetic construct
  misc_feature 1..20: note = PCR primer
  source 1..20: mol_type = other DNA
ggaatggctgcacctcacag

SEQ ID NO: 7 - DNA, 20 bp, synthetic construct
  misc_feature 1..20: note = PCR primer
  source 1..20: mol_type = other DNA
cggctgctaccacagtgatg

SEQ ID NO: 8 - DNA, 21 bp, synthetic construct
  misc_feature 1..21: note = PCR primer
  source 1..21: mol_type = other DNA
gaggctgtagctccccgttag

(19) United States
(12) Patent Application Publication
(10) Pub. No.: US 2024/0271104 A1
(43) Pub. Date: Aug. 15, 2024

(54) COLONIZATION OF SURFACES WITH BIOLOGICAL CELLS

(71) Applicant: Eberhard Karls Universität Tübingen Medizinische Fakultät, Tübingen (DE)

(72) Inventors: Meltem Avci-Adali, Kirchentellinsfurt (DE); Denis Canjuga, Tübingen (DE)

(21) Appl. No.: 18/603,977

(22) Filed: Mar. 13, 2024

Related U.S. Application Data
(63) Continuation of application No. PCT/EP22/72853, filed on Aug. 16, 2022.

(30) Foreign Application Priority Data
Sep. 14, 2021 (DE) 102021123779.3

(51) Int. Cl. C12N 5/071; C12N 11/06

(52) U.S. Cl. CPC C12N 5/0691; C12N 11/06

(57) ABSTRACT

The present invention relates to a method for producing a surface which can be colonized with biological cells, a device having a surface which can be colonized with biological cells, and a method for colonizing a surface with biological cells.

### CROSS-REFERENCES TO RELATED APPLICATIONS

The application is a continuation of international Patent Application No. PCT/EP2022/072853, filed on 16 Aug. 2022 and designating the United States of America, which has been published in German, and which claims priority from German patent application DE 10 2021 123 779.3 filed on 14 Sep. 2021. The entire contents of these prior applications are incorporated herein by reference.

### INCORPORATION BY REFERENCE FOR MATERIAL SUBMITTED ELECTRONICALLY

Incorporated by reference in its entirety is a computer-readable nucleotide/amino acid sequence listing identified as follows: 11,061 byte extensible Markup Language (.xml) file named “59955_Sequence_Listing.xml”; created on Mar. 7, 2024.

### FIELD OF THE INVENTION

The present invention relates to a method for producing a surface that can be colonized with biological cells, a device having a surface that can be colonized with biological cells, and a method for colonizing a surface with biological cells.

### BACKGROUND

Chronic obstructive pulmonary disease (COPD) is the third leading cause of death worldwide, and lung transplantation is the only curative treatment option for patients with end-stage lung diseases such as COPD, interstitial lung disease, cystic fibrosis and pulmonary edema. In 2017, only 77% of patients registered for a lung transplant in Germany were able to receive a lung, which means that 23% of these patients on the waiting list die each year. Even after a successful transplant, the median survival rate for lung recipients is only 5.3 years. There is therefore an urgent need for artificial lungs that can take over the function of the lungs for a longer period of time in order to bridge the gap until transplantation.

In severe respiratory failure, extracorporeal membrane oxygenation (ECMO) is performed by continuously pumping the patient's blood through a gas-permeable membrane oxygenator (artificial lung) that mimics the gas exchange process of the lungs. Standard blood oxygenators consist of bundles of microporous hollow fiber membranes (HFM), usually made of polypropylene (PP) or polymethylpentene (PMP), with a large surface area (˜2 m²) to achieve the clinically required transport rates for O₂ and CO₂. In most artificial lungs, oxygen flows through the lumina of the hollow fibers and blood flows through the spaces in the hollow fiber bundle.

Despite the treatment of patients during ECMO with anticoagulant drugs, mostly heparin, the foreign material surface of the artificial lungs still leads to activation of the coagulation and complement system and makes their long-term use difficult, so that they can currently only be used for a very limited period of days to a few weeks [Gulack, B. C., S. A. Hirji, and M. G. Hartwig, Bridge to lung transplantation and rescue post-transplant: the expanding role of extracorporeal membrane oxygenation. J Thorac Dis, 2014. 6(8): pp. 1070-1079]. For decades, intensive research has been conducted to improve the hemocompatibility of blood-contacting artificial devices [de Mel, A., B. G. Cousins, and A. M. Seifalian, Surface modification of biomaterials: a quest for blood compatibility. Int J Biomater, 2012. 2012: p. 707863; Wendel, H. P. and G. Ziemer, Coating-techniques to improve the hemo-compatibility of artificial devices used for extracorporeal circulation. Eur J Cardiothorac Surg, 1999. 16(3): pp. 342-350]. So far, blood-contacting oxygenator surfaces have been coated with e.g. phosphorylcholine [Pieri, M., et al, A new phosphorylcholine-coated polymethylpentene oxygenator for extracorporeal membrane oxygenation: a preliminary experience. Perfusion, 2013. 28(2): pp. 132-137], Heparin [Pappalardo, F., et al, Bioline heparin-coated ECMO with bivalirudin anticoagulation in a patient with acute heparin-induced thrombocytopenia: the immune reaction appeared to continue unabated. Perfusion, 2009. 24(2): p. 135-137] or poly-2-methoxyethyl acrylate (PMEA) [Eisses, M. J., et al, Effect of polymer coating (poly 2-methoxyethyl acrylate) of the oxygenator on hemostatic markers during cardiopulmonary bypass in children. J Cardiothorac Vasc Anesth, 2007. 21(1): pp. 28-34].

The coating of surfaces with polydopamine is known, for example, from CN 110545754, and the coating with polymers from U.S. Pat. No. 6,309,660. However, neither approach has proven successful in practice.

The most common approach is to coat the surfaces with heparin, as described, for example, in WO 2020/190214. Over time, however, the heparin can detach from the surface and lead to the formation of thrombi. In addition to complications such as bleeding, long-term exposure to heparin carries the risk of the patient developing heparin-induced thrombocytopenia (HIT). This involves the formation of antibodies against the heparin and platelet factor 4 (PF4) complex, which can lead to severe thromboembolic complications due to strong platelet activation, increased thrombin formation and activation.

As described in detail in a review article [Avci-Adali, M., et al, Current concepts and new developments for autologous in vivo endothelialization of biomaterials for intravascular applications. European cells & materials, 2011. 21: p. 157-176], various scavenger molecules and strategies have been used for endothelialization of blood-contacting implants. So far, HFM with heparin/albumin [Wiegmann, B., et al, Developing a biohybrid lung—sufficient endothelialization of poly-4-methly-1-pentene gas exchange hollow-fiber membranes. J Mech Behav Biomed Mater, 2016. 60: p. 301-311], cRGD [Moller, L., et al, Towards a biocompatible artificial lung: Covalent functionalization of poly(4-methylpent-1-ene) (TPX) with cRGD pentapeptide. Beilstein Journal of Organic Chemistry, 2013. 9: pp. 270-277] or fibronectin [Cornelissen, C. G., et al, Fibronectin coating of oxygenator membranes enhances endothelial cell attachment. Biomed Eng Online, 2013. 12: p. 7] to support endothelialization. However, these strategies have several limitations, e.g. coating the surfaces with fibronectin may lead to non-specific attachment of unwanted cells, and the non-endothelialized areas may activate platelets and lead to thrombosis. The use of cRGD to bind endothelial cells (“ECs”) is also non-specific. Several cells, including platelets, have cRGD-binding integrins on their surface, which can lead to non-specific adhesion of cells to the blood-contacting surface. Thus, an efficient strategy is still needed to endothelialize blood-contacting areas with ECs and improve the hemocompatibility of the artificial lungs.

### SUMMARY

Against this background, it is an object of the present invention to provide a method for producing a surface which can be colonized with biological cells, in which the disadvantages of the prior art are avoided or at least reduced. In particular, such a surface is to be provided which can be colonized with biological cells in such a way that a high degree of biocompatibility is achieved.

This object is solved by a method for preparing a surface which can be colonized with biological cells, which comprises the following steps:
1. providing a surface comprising hydroxyl groups (—OH),
2. silanization of the surface containing hydroxyl groups,
3. conjugation of a reactant reacting with azides in a copper-free click reaction to the silanized surface.

According to the invention, a “surface” comprises any surface that is to be colonized with biological cells, e.g. endothelial cells. This includes, but is not limited to, surfaces of medical products or medical devices, such as artificial lungs of oxygenators, e.g. the walls of membranes where gas exchange takes place. Plastic or metal surfaces are also covered by the invention.

According to the invention, “biological cells” is understood to mean any type of cell, such as endothelial cells, but also stem cells, including mesenchymal stem cells, as well as progenitor cells thereof or cells derived therefrom, such as endothelial progenitor cells (EPCs) or endothelial cells derived from induced pluripotent stem cells (“iPSC-derived endothelial cells”).

The provision of hydroxyl groups (—OH) on the surfaces in step 1 of the method according to the invention can be carried out by methods known to the person skilled in the art. These include, but are not limited to, the treatment of the surface with oxygen plasma. Thus, hydroxyl groups can be generated on the surface by this treatment of membranes, for example polymethyl-pentene hollow fiber membranes.

“Silanization” in step 2 of the method according to the invention is understood to mean the chemical bonding of a silane compound to the surface. This can be achieved, for example, but not exclusively, by a reaction of the free hydroxyl groups with 3-aminopropyltriethoxysilane (APTES).

In step 3 of the method according to the invention, the reactant, which can react with azides in a copper-free click reaction, such as, for example, but not exclusively, dibenzocyclooctyne (DBCO), is conjugated to the silanized surface using methods known to the skilled person.

According to the invention, a “click reaction”, synonymously also referred to as “click chemistry”, is understood to mean a reaction between azide and alkyne to provide a 1,5-disubstituted 1,2,3-triazole. According to the invention, this takes place without the use of copper or other cytotoxic catalysts under physiological conditions, e.g. in a cell culture medium. This represents a departure from the “click reactions” used in the prior art. For example, copper-catalyzed azide-alkyne cycloadditions (CuAAC) are predominantly carried out there. However, this method is unsuitable according to the invention due to the use of copper, since copper damages the biological cells. The invention provides a remedy by being copper-free.

The surfaces prepared according to the invention are now able to react with azides in a copper-free click reaction and thereby covalently bind structures that expose azide groups (N₃) to the surface. For example, endothelial cells can be functionalized in such a way that they have glycoproteins with azide groups on their surfaces. These groups can be covalently conjugated to the reactant, such as DBCO, in a copper-free click reaction.

The method according to the invention thus provides surfaces that can be efficiently and specifically colonized with azide-modified biological cells. Since this reaction is very specific, only those biological cells that expose azide groups are bound to the surface. This prevents the non-specific binding of unwanted cell populations. This enables efficient colonization of the surfaces with endothelial cells, for example, in order to prevent the surfaces from being recognized as “foreign”. The method according to the invention can also be used to bind the patient's own endothelial progenitor cells (EPCs) or endothelial cells derived from induced pluripotent stem cells (“iPSC-derived endothelial cells”) to desired surfaces of blood-contacting materials.

The problem underlying the invention is thus completely solved.

In an embodiment the method according to the invention comprises the following further step: 4. incubation of the surface with biological cells which have azide groups (—N₃) on their surface, under conditions which permit conjugation of the azide group with the reactant in a copper-free click reaction.

In another embodiment of the invention, the reactant of step 3 is selected from the group consisting of: cycloalkyne, cycloalkyne ester, phoshin and phosphine ester.

This measure has the advantage that reactants are used which are suitable for reacting with azides in a copper-free click reaction and are thus particularly functional according to the invention. Phosphine groups react with the azide groups in a so-called Staudinger reaction. This reaction shows slow reaction kinetics of about 10⁻³ M⁻¹s⁻¹. Cycloalkynes react with the azide groups in a so-called ‘strain-promoted azide-alkyne cycloaddition reaction’ (SPAAC). The 2nd order reaction has a moderate rate constant of 10⁻²-1 M⁻¹s⁻¹.

In a further embodiment of the invention, the cycloalkyne dibenzocyclooctyne (DBCO) and/or the cycloalkyne ester DBCO-PEG₄-NHS ester is used as reactant.

This measure has the advantage that such reactants are used which, according to the inventors' knowledge, have proven to be particularly suitable. The suitability of DBCO according to the invention was particularly surprising. For example, the prior art describes possible uses of DBCO to functionalize molecules, generally proteins: see, e.g., Wallrodt, S., et al, Bioorthogonally Functionalized NAD(+) Analogues for In-Cell Visualization of Poly(ADPRibose) Formation. Angew Chem Int Ed Engl, 2016. 55(27): pp. 7660-7664; Laughlin, S. T., et al, In vivo imaging of membrane-associated glycans in developing zebrafish. Science, 2008. 320(5876): P. 664-667; Xie, R., et al, In vivo metabolic labeling of sialoglycans in the mouse brain by using a liposome-assisted bioorthogonal reporter strategy. Proceedings of the National Academy of Sciences of the United States of America, 2016. 113(19): pp. 5173-5178; Koo, H., et al, Bioorthogonal Copper-Free Click Chemistry In Vivo for Tumor-Targeted Delivery of Nano-particles. Angewandte Chemie-International Edition, 2012. 51(47): p. 11836-11840. However, conjugation of DBCO to a sialized surface for the purpose of subsequent colonization with biological cells has not yet been described in the state of the art.

In an embodiment of the method according to the invention, the surface having hydroxyl groups is provided in step 1 by oxygen plasma treatment of a surface.

This measure has the advantage that a process is used which is particularly suitable for generating hydroxyl groups on the surface.

In an embodiment of the invention, the surface is that of a medical device, preferably selected from the group consisting of: artificial lung, oxygenator, artificial kidney, prosthesis, vascular prosthesis, stent, artificial heart.

This measure has the advantage that the method is used where the colonization of surfaces with biological cells is particularly important for the functionality and biocompatibility of the devices. When used in patients, this can prevent the device from being recognized as “foreign”.

In a further embodiment of the invention, the surface is a surface of a hollow fiber membrane (HFM), preferably a polymethylpentene or polypropylene HFM.

This measure has the advantage that the method can be used for surfaces that come into contact with blood, which are particularly relevant in practice and are used, for example, in oxygenators.

In a still further embodiment of the invention, the biological cells are endothelial cells.

With this measure, cells that are of particular importance for the functionality of important medical devices, such as artificial lungs, can be bound to the surfaces.

In an embodiment of the method according to the invention, the biological cells have azide groups (—N₃) on their surface.

This measure advantageously creates the technical conditions for the cells to be bound to the surface produced according to the invention in a copper-free click reaction.

Another subject-matter of the invention relates to a device with a surface that can be colonized with biological cells, preferably an artificial lung, an oxygenator, an artificial kidney, a prosthesis, a vascular prosthesis, a stent, an artificial heart or a hollow fiber membrane (HFM) such as a polymethylpentene or polypropylene HFM, wherein the surface comprises a reactant reacting with azides in a copper-free click reaction. Preferably, the surface colonizable with biological cells was obtained by the method according to the invention.

The features, properties, advantages, and embodiments of the preparation method according to the invention also apply in a corresponding manner to the device according to the invention.

Another subject-matter of the present invention relates to a method for colonizing a surface with biological cells, comprising the following steps:
1. providing a surface comprising a reactant reacting with azides in a copper-free click reaction,
2. incubating the surface with biological cells having azide groups (—N₃) on their surface under conditions that allow conjugation of the azide group with the reactant in a copper-free click reaction.

In an embodiment of the colonization method according to the invention, the surface comprising a reactant reacting with azides in a copper-free click reaction was obtained by the preparation method according to the invention.

In a further embodiment of the colonization method according to the invention, the biological cells having azide groups (—N₃) on their surface were obtained by incubating biological cells with an azide sugar preferably selected from the group consisting of: Ac4ManNAz (N-azidoacetylmannos-amine-tetraacylated), N-azidoacetylglucosamine-tetraacylated (Ac4GIcNAz), N-azidoacetylgalactosamine-tetraacylated (Ac4GaINAz) and other azide-functionalized glycoconjugates.

This measure advantageously produces biological cells that expose azide groups (—N₃) on their surface. The specified azide sugars are particularly suitable for the functionalization of the cells. The azide sugars are taken up into the cells like their natural analogs and incorporated into glycoproteins. As a result, the cells have glycoproteins on the cell surface that are provided with azide groups (—N₃). These groups can be covalently conjugated to the surface using click chemistry. Incubation of the modified cells with the modified HFMs results in a bio-orthogonal reaction, the alkyne group of the reactant or DBCO reacts with the azide on the surface of the cells. This happens very specifically, so that only modified cells with azide molecules can bind to the reactants on the membrane. Binding of the cells to the membrane and further incubation in cell culture medium leads to proliferation and ultimately to colonization of the membrane with a confluent monolayer of cells.

In a further embodiment of the colonization method according to the invention, the biological cells are endothelial cells.

With this measure, the surfaces are colonized with cells that are of particular significance for the functionality of important medical surfaces or devices. Particularly suitable are primary endothelial cells isolated from umbilical cord blood, so-called Human Umbilical Vein Endothelial Cells (HUVECs), which are cultivated in VascuLife® EnGS Endothelial Medium Complete Kit until endothelialization. In one embodiment, colonization with the endothelial cells can take place in EGM-2 Endothelial Cell Growth Medium-2 BulletKit. Polymethylpentene HFMs (PMP, OXYPLUS, 3M Membrana, Wuppertal, Germany), APTES, methanol, toluene (Sigma-Aldrich, Darmstadt), DBCO-PEG₄-NHS ester (Jena Bioscience, Jena, Germany), Dulbe-cco's phosphate-buffered saline (DPBS), (Invitrogen), N-azidoacetylmannosamine-tetraacylated (Ac4ManNAz, Sigma Aldrich, Germany), DMSO (Sigma-Aldrich).

The features, properties, advantages and embodiments of the manufacturing process according to the invention also apply in a corresponding manner to the colonization process according to the invention.

It is understood that the above-mentioned features and those to be explained below can be used not only in the combinations indicated in each case, but also in other combinations or on their own, without departing from the scope of the present invention.

The invention will now be explained in more detail with reference to examples. The features mentioned therein are considered to belong to the invention not only in relation to the specific example, but also in isolated form.

Reference is made to the accompanying figures.

### BRIEF DESCRIPTION OF THE DRAWINGS

FIGS. 1A-1D: Schematic representation of the functionalization of the PMP hollow fiber membranes (HFMs). 1A) Generation of hydroxyl groups by O₂ plasma treatment. 1B) Silanization of the plasma-treated HFMs with APTES. 1C) Introduction of cyclooctyne groups by covalent binding of DBCO-PEG₄-NHS esters. 1D) Click reaction of modified HFMs with metabolically altered endothelial cells.

FIGS. 2A-2C: Detection of the generated functional groups on PMP hollow fiber membranes (HFMs). (2A) To detect the amino groups after APTES treatment of the HFMs, the HFMs were stained with methyl orange, and the absorbance of surface-bound and eluted methyl orange was measured with a spectrophotometer. Untreated and O₂ plasma-treated HFMs were used as negative controls. (n=3). (2B) To detect DBCO groups on the surface of untreated HFMs treated with APTES or DBCO-PEG-NHS ester, the HFMs were incubated with Cy3-azide. The conjugated Cy3-azide was detected with a fluorescence reader (n=3) or (2C) by fluorescence microscopy. Data are expressed as mean±SEM. Statistical analysis was performed using one-way ANOVA followed by Tukey's comparison test. (*** p<0.001, **** p<0.0001).

FIG. 3: Result of the SEM analyses. There are no visual differences on the surface of the HFM treated with APTES only or APTES and DBCO-PEG₄-NHS ester compared to untreated HFM.

FIGS. 4A-4C: Analysis of the impermeability of HFM membranes after surface functionalization. 4A) To measure possible leakage, HFMs 4B) untreated (left) and O₂ plasma-treated (right) and 4C) APTES-(left) and DBCO-(right) functionalized membranes were placed in a closed circuit filled with 0.1% toluidine blue solution and incubated under a constant pressure of 250 mmHg±10 mmHg. No leakage was detected after 3 hours, demonstrating the maintenance of membrane integrity. Representative images are shown (n=3).

FIG. 5: Analysis of the presence of N₃ groups on the surface of HUVECs after removal of Ac4ManNAz from the cell culture medium. 2×10⁵ HUVECs were incubated for 48 h with 50 UM Ac4ManNAz and without. The cells were then washed and the medium was replaced with cell culture medium without Ac4ManNAz. After 0, 2, 4 and 24 h, cells were incubated with 5 μM DBCO-Sulfo-Cy3 for 1 h and flow cytometric analyses were performed after washing with DPBS. The results are expressed as mean+SD (n=3). Statistical analysis was performed using two-way ANOVA followed by Bonferroni multiple comparison test (** p<0.01, *** p<0.001, ns: not significant).

FIGS. 6A-6C: Metabolic labeling of HUVECs with Ac4ManNAz and analysis of cell viability. 2×10⁵ HUVECs were incubated for 48 h with and without 50 UM Ac4ManNAz. A) The cells were incubated for 1 h with 5 μM DBCO-Sulfo-Cy3. After washing the cells with DPBS and staining the cytoskeleton with ActinGreen and the nuclei with DAPI, fluorescence micrographs were taken. B) Cells were incubated with 5 μM DBCO-Sulfo-Cy3 for 1 hour and flow cytometric analyses were performed after washing with DPBS. Results are expressed as mean±SD (n=3). C) Cell viability was determined using the PrestoBlue assay. The results are expressed as mean+SEM (n=3). Statistical analysis was performed using a two-tailed paired t-test. (** p<0.01, ns: not significant).

FIGS. 7A-7D: Analysis of endothelialization of modified hollow fiber membranes (HFMs). Untreated, APTES- or DBCO-functionalized HFMs were incubated with HUVECs. For this purpose, 5.2×10⁶ HUVECs without (w/o) or with metabolic labeling with Ac4ManNAz were dynamically incubated for 24 h and then cultured for 24 h under static conditions. 7A) The attached cells were visualized by calcein AM staining and fluorescence microscopy. Representative images of three experiments are shown. 7B) Quantification of bound HUVECs on HFMs using a fluorescence reader. The results are shown as mean+SEM (n=3). Statistical analysis was performed using one-way ANOVA followed by Tu-key's Multiple Comparison Test. (*p<0.05, ** p<0.01). 7C) Detection of cell-cell contacts of HUVECs attached to the DBCO-coated membranes by staining with anti-human VE-cadherin (green) and DAPI as nuclear staining by fluorescence microscopy. 7D) Detection of cell-cell contacts of HUVECs bound to the DBCO-coated membranes by staining with anti-human VE-cadherin antibody (green) and ActinRed cytoskeleton staining using fluorescence microscopy.

FIG. 8: Analysis of the response of the endothelialized surface to an inflammatory stimulus using qRT-PCR. Endothelialized HFM were treated with 50 ng/ml TNF-α, and the expression of adhesion molecules (E-selectin, VCAM-1 and ICAM-1) was determined by qRT-PCR. The mRNA levels were normalized to the GAPDH mRNA levels, and the results are expressed as mean+SEM relative to the expression levels in the negative control (endothelialized HFM without TNF-α stimulation). Differences were determined using a one-sided t-test. (n=3). (*p<0.05, *** p<0.001, **** p<0.0001).

FIG. 9: Determination of blood cell count after incubation of human blood with uncoated, DBCO-coated or endothelialized HFM. Blood samples without HFM were used as negative control. The blood was incubated for 90 minutes at 37° C. The number of white blood cells, red blood cells and platelets was determined using a cell counter. In addition, activated platelets were detected by staining with an anti-human CD62P antibody and by flow cytometry. The data are given as mean+SD. Statistical analysis was performed by one-way ANOVA followed by Bonferroni multiple comparison test. (*p<0.05, ** p<0.01, *** p<0.001).

FIG. 10: Scanning electron microscopy (SEM) images of uncoated, DBCO-coated or endothelialized HFMs 90 minutes after incubation with human blood or endothelialized HFMs without blood contact. (n=3).

FIG. 11: Analysis of the hemocompatibility of uncoated, DBCO-coated or endothelialized HFMs. After 90 min incubation with fresh human blood, activation of coagulation (thrombin-antithrombin III complex (TAT)), platelets (β-thromboglobulin), complement system (sC5b-9) and inflammation (PMN elastase) were determined by ELISA (n=4). The data are given as mean+SD. Statistical analysis was performed using one-way ANOVA followed by Bonferroni multiple comparison test. (** p<0.01, *** p<0.001, and **** p<0.0001).

FIG. 12: Analysis of the inflammatory status of the endothelialized surface of HFMs by qRT-PCR. Endothelialized HFMs were incubated with human blood at 37° C. for 90 minutes, and the expression of adhesion molecules (E-selectin, VCAM-1 and ICAM-1) was determined by qRT-PCR. The mRNA levels were normalized to the GAPDH mRNA levels, and the results are expressed as mean+SEM relative to the expression levels in the control (HUVECs on cell culture plate). Differences were determined by one-way ANOVA followed by Bonferroni multiple comparison test. (n=3). (*p<0.05, ** p<0.01, *** p<0.001).

### EXAMPLES

### 1. Material and Methods

### 1.1 Cultivation of Human Umbilical Vein Endothelial Cells (HUVECs)

HUVECs (ATCC®, Manassas, USA) were seeded in T75 cell culture flasks coated with 0.1% gelatin and cultured at 37° C. and 5% CO₂ in Vasculife® EnGS EC culture medium (CellSystems, Troisdorf, Germany) with VascuLife EnGS LifeFactors Kit, 50 mg/ml gentamicin and 0.05 mg/ml amphotericin B (Thermo Fisher Scientific, Waltham, USA). The medium was changed every 3 days. After reaching 80% confluence, the cells were detached with trypsin/EDTA (0.04%/0.03%, PromoCell, Heidelberg, Germany).

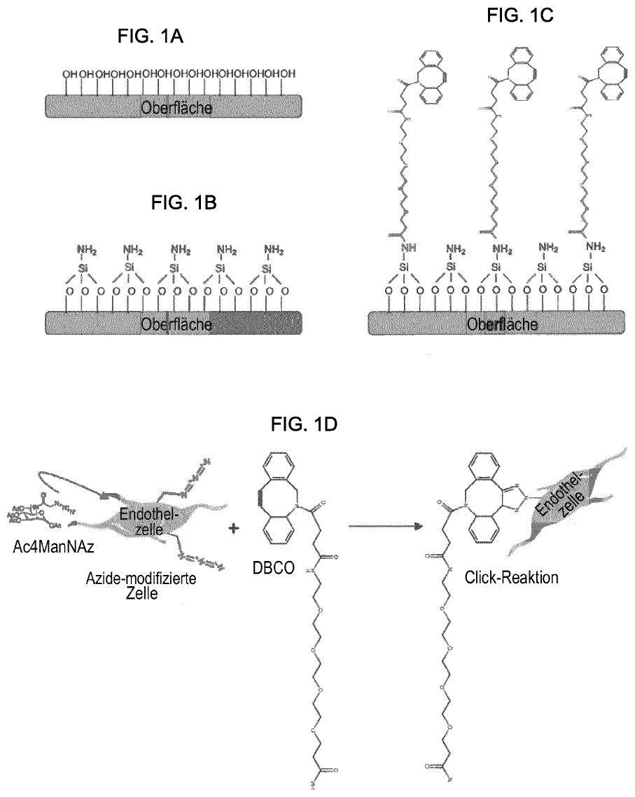

The surface of polymethylpentene HFMs (PMP, OXYPLUS, 3M Membrana, Wuppertal, Germany) was coated in a three-step process (FIGS. 1A-1D). First, the surface was treated with O₂ plasma, and silanization was performed in the second step. In the third step, dibenzylcyclooctyne-PEG4-NHS ester (DBCO-PEG₄-NHS ester) was conjugated to the silanized surface. For this purpose, PMP membranes were cut to a size of 3×3 cm and hydroxyl groups were generated by treatment with O₂ plasma (Denta Plas®, Diener electronic, Ebhausen, Germany) for 30 min at a pressure of 0.3 mbar (+0.20 mbar) and a power of 80% (+5%). The HFMs were then incubated in toluene (Sigma-Aldrich, Darmstadt, Germany) containing 2% (3-aminopropyl)triethoxysilane (APTES, Sigma-Aldrich) for 30 min on a shaker at 20 rpm. The membranes were then sonicated in 100% toluene, 50% toluene/50% methanol and 100% methanol for 2 minutes each to remove unbound or weakly bound APTES. After drying, the membranes were incubated in 400 μM DBCO-PEG₄-NHS ester (Jena Bioscience, Jena, Germany) with Dulbecco's phosphate buffered saline (DPBS) (Invitrogen, Carlsbad, USA) for 30 minutes to introduce cyclooctyne groups and washed three times with fresh DPBS.

### 1.3 Quantification of the Functional Groups Formed on the HFM Coating

Detection of Amino Groups after APTES Treatment

To detect the amino groups on the HFM surface, the membranes were incubated in 0.5 mM methyl orange (Sigma-Aldrich, Darmstadt, Germany) for 5 hours at 37° C. on a shaker, followed by washing three times with 1 mM HCl solution. Desorption of bound methyl orange from the HFM surface was performed with 0.4 ml of a 1 mM NaOH solution overnight at 37° C. on a shaker. To detect methyl orange, 100 μl of the desorption solution was added in triplicate to a 96-well plate. The absorbance was measured at 465 nm using a microplate reader (Eon Synergy 2, Bio Tek Instruments Inc., Winooski USA).

Detection of DBCO groups after conjugation of DBCO-PEG₄-NHS ester

After incubation with DBCO-PEG₄-NHS ester, the HFMs were incubated in DPBS without Ca²⁺/Mg²⁺ for 1 hour with 80 μg/ml Cy3 azide (Sigma-Aldrich) to bind the Cy3 azide to the DBCO-functionalized HFM surface. The HFMs were then washed five times with 100% ethanol and the staining of the membranes was detected by fluorescence microscopy (Axiovert 135, Carl Zeiss A G, Oberkochen, Germany). In addition, the fluorescence intensity on the membranes was quantified using a microplate reader (Mithras 940, Berthold Technologies, Bad Wildbad, Germany) at an excitation wavelength of 500 nm and an emission wavelength of 600 nm. HFM without treatment and only with APTES treatment were used as controls.

1.4 Metabolic Labeling of HUVECs with Azide Groups and Detection of Azide Labeling

To functionalize the cell surface of HUVECs with azide groups, 2×10⁵ HUVECs per well of a 6-well plate were seeded and treated with or without 50 μM N-azidoacetylmannosamine-tetraacylated (Ac4ManNAz, Sigma Aldrich) in 2 ml medium for 48 h at 37° C. with 5% CO₂. To detect the formed azido groups, the cells were washed with DPBS without Ca²⁺/Mg²⁺ and incubated with 5 μM DBCO-Sulfo-Cy3 (Jena Bioscience, Germany) in DPBS with Ca²⁺/Mg²⁺ for 1 h at 37° C. and 5% CO₂. Cells were washed three times with 10 ml DPBS, detached with trypsin/EDTA (0.04%/0.03%) and centrifuged at 300×g for 5 min. The cell pellet was resuspended in 0.5 ml DPBS, and Cy3 labeling of 10,000 cells was analyzed by flow cytometry (FACS SCAN, BD, Heidelberg, Germany). In addition to the flow cytometric analyses, cells were stained for 30 minutes with ActinGreen™ 488 ReadyP-robes® reagent (Invitrogen) for the cytoskeleton and for 5 minutes with 0.2 μg/ml DAPI (Sigma Aldrich) to visualize the nuclei.

1.5 Analysis of Cell Viability after Metabolic Labeling of HUVECs with Ac4ManNAz

The influence of metabolic labeling on the viability of HUVECs was investigated 48 hours after labeling with 50 UM Ac4ManNAz using the PrestoBlue assay (Invitrogen, Carlsbad, USA). For this purpose, 500 μL of 1× Presto Blue cell viability reagent was added to each well of a 6-well plate and incubated for 1.5 h at 37° C. The fluorescence intensity of 100 μL supernatant was measured in triplicate at an excitation wavelength of 530 nm and an emission wavelength of 600 nm using a multimode microplate reader (Mithras LB 940, Berthold Technologies, Bad Wildbad, Germany).

### 1.6 Clicking of Endothelial Cells on HFM Using Copper-Free Click Chemistry

DBCO-coated and uncoated PMP membranes (3×3 cm) were incubated with 5.2×10⁶ Ac4ManNAz-labeled HUVECs in 13 ml EGM™-2 endothelial cell growth medium BulletKit™ (Lonza, Basel, Switzerland). Incubation was performed for 24 hours at 10 rpm with a rotator (Phoenix Instrument, Garbsen, Germany) in a humidified incubator with 5% CO₂ at 37° C. After rotational incubation, the membranes were placed in each well of a 6-well plate and further incubated for 1 or 5 days under static conditions.

### 1.7 Analysis of Endothelialization

The HUVECs adhering to the membranes were stained with 250 ng/ml calcein acetoxymethyl ester (Calcein-AM; Invitrogen) for 10 minutes at 37° C. The stained cells were detected by fluorescence microscopy (Axio 135, Carl Zeiss A G, Oberkochen, Germany). In addition, the fluorescence signal on the surface of the HFMs before and after calcein-AM staining was detected at an excitation wavelength of 494 nm and an emission wavelength of 517 nm using a multimode microplate reader (Mithras LB 940, Berthold Technologies).

In addition, the formation of a confluent endothelial cell monolayer and the presence of cell-cell contacts were detected by staining the cells with an anti-human VE-cadherin antibody. For this purpose, the endothelialized membranes coated with DBCO-PEG₄-NHS ester were washed with DPBS and fixed in 4% paraformaldehyde (v/v) for 10 minutes at RT. After washing with DPBS, blocking was performed with Tris-buffered saline (50 mM Tris base, 150 mM sodium chloride, pH 7.5) containing 5% goat serum (Thermo Fisher Scientific, Waltham, USA) for 30 minutes at RT. The membranes were washed with DPBS and incubated with the 1:100 diluted primary antibody mouse anti-human VE-cadherin (CD144 (16B1), eBioscience, Invitrogen, USA) in DPBS containing 1% bovine serum albumin (BSA) for 1 h at RT. The membranes were then washed three times with DPBS and incubated with 1:400 diluted Alexa Fluor 488-labeled goat anti-mouse IgG (H+L) cross-adsorbed secondary antibody (ThermoFisher, USA) for 1 h in PBS containing 1% BSA. After washing three times with DPBS, the cell nuclei were stained for 5 min with DAPI (Sigma Aldrich, Darmstadt, Germany) diluted 1:2000 in DPBS. As a negative control, the membranes were also incubated with an isotype control antibody. The stained cells were detected by fluorescence microscopy (Axio 135, Carl Zeiss A G, Aoberkochen, Germany).

### 1.8 Reactivity of the Endothelium to HFMs to the Inflammatory Stimulus

Endothelialized HFMs were incubated for 4 hours without or with 50 ng/mL tumor necrosis factor α (TNF-α, Sigma-Aldrich) in EGM™-2 endothelial cell growth medium without hydrocortisone. After incubation, HFMs were washed twice with DPBS and total RNA was isolated. Expression of the activation markers, endothelial leukocyte adhesion molecule 1 (E-selectin), vascular cell adhesion molecule 1 (VCAM-1) and intercellular adhesion molecule 1 (ICAM-1), was detected by qRT-PCR. The mRNA levels were normalized to the GAPDH mRNA levels, and the results were presented relative to the expression levels in endothelialized HFM without TNF-α stimulation.

1.9 qRT-PCR

Total RNA of the cells was isolated using the Aurum Total RNA Mini Kit (Bio-Rad, Munich, Germany) according to the manufacturer's instructions. 300 ng of RNA was reverse transcribed into DNA copies (cDNA) using the iScript Kit (Bio-Rad). Amplification of transcripts was performed with the primers listed in Table 1 at a final concentration of 300 nM under the following conditions: one cycle of 3 min at 95° C., followed by 40 cycles of 95° C. for 15 s and 72° C. for 10 s. After 40 cycles, melting was performed. After 40 cycles, a melting curve analysis was performed to detect the specific amplicons. Real-time qRT-PCR reactions were performed in triplicate with a total volume of 15 μL per well in a CFX Connect Real-Time PCR Detection System (Bio-Rad) using IQ SYBR Green Supermix (Bio-Rad). The constitutively expressed GAPDH (glyceraldehyde-3-phosphate dehydrogenase) was used as housekeeping gene. The detected mRNA levels of each gene were normalized to GAPDH, and the results were expressed relative to the control mRNA levels.

TABLE 1
Primers used for qRT-PCR analysis.

| Gene | Forward primer (5′→3′) | Backward primer (5′→3′) |
|---|---|---|
| GAPDH | TCAACAGCGACACCCACTCC | TGAGGTCCACCACCCTGTTG |
| | (SEQ ID NO: 1) | (SEQ ID NO: 5) |
| E- | GCCCAGAGCCTTCAGTGTACC | GGAATGGCTGCACCTCACAG |
| selectin | (SEQ ID NO: 2) | (SEQ ID NO: 6) |
| ICAM-1 | CTTGAGGGCACCTACCTCTGTC | CGGCTGCTACCACAGTGATG |
| | (SEQ ID NO: 3) | (SEQ ID NO: 7) |
| VCAM-1 | ACACTTTATGTCAATGTTGCCCC | GAGGCTGTAGCTCCCCGTTAG |
| | (SEQ ID NO: 4) | (SEQ ID NO: 8) |

### 1.10 Blood Collection

Human whole blood was collected with a Safety-Multyfly® 20 Gx3/4 TW needle (Sarstedt, Nümbrecht, Germany) from the antecubital vein of healthy subjects (n=4) and filled into 9 mL monovettes (Sarstedt) with 1 IU/mL sodium heparin (Ratiopharm, Ulm, Germany). The ethics committee of the University of Tübingen approved the blood collection and all subjects gave their written consent.

### 1.11 Performance of Hemocompatibility Analyses

Incubation of unmodified, DBCO-coated or endothelialized HFM (3×3 cm) with human blood samples was performed with 9 mL heparinized human whole blood in polypropylene round-bottom tubes (14 mL, BD Biosciences, Germany) under dynamic conditions for 90 minutes at 37° C. and 30 rpm. As a negative control, the tubes were filled with the same amount of fresh heparinized blood without any material. After 90 minutes of dynamic incubation, the blood samples were collected in tubes containing ethylenediaminetetraacetic acid (1.6 mg/mL, EDTA, Sarstedt) for the analysis of complement activation and detection of cell number. Tubes containing (0.3 mL citrate solution/3 mL blood, 0.106 M C₆H₅Na₃O₇×2H₂O, Sartstedt) were used to detect PMN elastase and TAT. For β-TG analysis, blood was transferred to 2.7 mL CTAD tubes containing 270 μL of 0.109 M CTAD solution containing buffered sodium citrate, theophylline, adenosine and dipyridamole (BD Vacutainer CTAD, Becton-Dickinson GmbH, Heidelberg, Germany) and stored on ice for 15 minutes. The EDTA and CTAD preparations were centrifuged at 2500×g for 20 minutes at 4° C. The citrated blood preparations were centrifuged at 1800×g for 18 min at RT. The blood plasma of each sample was flash frozen in liquid nitrogen and stored at −80° C. until further analysis.

### Analysis of the Blood Cell Count

The number of erythrocytes, leukocytes and thrombocytes was measured in the collected blood samples using an automatic cell counting system (ABX Micros 60, HORIBA Medical, Minami-ku, Kyoto, Japan).

### Analysis of Activation Markers in Blood Plasma

Commercially available enzyme-linked immunosorbent assay (ELISA) kits were used to determine changes in coagulation (thrombin-antithrombin III complex (TAT)), the complement system (sC5b-9) and activation of platelets (β-thromboglobulin (β-TG)) and neutrophils according to the manufacturer's instructions. Therefore, sC5b-9 (MicroVue™ Complement, Quidel Germany GmbH & AnDiaTec Division, Kornwestheim, Germany), TAT (Enzygnost®) TAT micro, Siemens Healthcare, Erlangen, Germany), polymorphonuclear (PMN) elastase (Demeditec Diagnostics, Kiel, Germany) and β-TG (Asserachrom® β-TG, Diagnostica Stago, Parsippany, NJ, USA) ELISAs were used.

### Detection of Activated Platelets

Uncoated, DBCO-coated or endothelialized HFMs were incubated with blood as described above. After incubation of the HFMs with human blood for 90 minutes, the blood was collected and diluted 1:5 with DPBS. Samples were incubated for 30 minutes at RT with 10 μl mouse anti-human CD41-FITC antibody (Company) to detect platelets and 10 μl mouse anti-human CD62P-PE antibody (BD Biosciences, Heidelberg, Germany) to detect platelets expressing P-selectin (CD62P), indicating platelet activation. Samples were fixed with 0.5% paraformaldehyde and platelet activation was analyzed by flow cytometry.

### Scanning Electron Microscopic (SEM) Analyses of the HFM Surfaces

After incubation of unmodified, DBCO-coated and endothelialized HFMs with blood, the HFMs were rinsed with 0.9% saline (Fresenius Kabi, Bad Homburg, Germany) and fixed in 2.5% glutaraldehyde (Sigma Aldrich, Darmstadt, Germany) in DPBS for 24 h. After the fixation step, the samples were washed with DPBS for 15 min at RT. After the fixation step, the samples were washed with DPBS for 15 min at RT and then dehydrated in 15 min steps at RT with an ascending ethanol series (40%-100% ethanol; Merck-Millipore, Darmstadt, Germany). Samples were then dried in a critical point dryer (Polaron E3100, Quorum Technologies ltd, East Sussex, United Kingdom) and sputter-coated with gold-palladium particles (Baltec SCD 050, Bal-Tec AG, Balzers, Liechtenstein). Imaging was performed using SEM (EVO LS 10, Carl Zeiss Microscopy GmbH, Jena, Germany) at 20×, 500×, 1000× and 5000× magnification.

### Analysis of the Inflammatory Status of the Endothelium on HFMs

After endothelialization of HFMs, total RNA was isolated from endothelial cells without blood incubation and after 90 minutes of blood incubation. For this purpose, HFMs were washed twice with DPBS prior to RNA isolation. As a control, the RNA was also isolated from HUVECs cultivated on 6-well plates. The expression of the activation markers E-selectin, VCAM-1 and ICAM-1 was detected by qRT-PCR. The mRNA levels were normalized to the GAPDH mRNA levels, and the results were presented in relation to the expression levels in HUVECs cultured on cell culture plates.

### 1.14 Statistical Analysis

Data are expressed as mean±standard deviation (SD) or mean±standard error of the mean (SEM). A two-tailed paired t-test was used to compare the mean values between two groups. For comparison of means between more than two groups, a one-way analysis of variance (ANOVA) with Bonferroni's multiple comparison test was performed. All statistical analyses were performed using GraphPad Prism 9.0.2 software (GraphPad Software, Inc., La Jolla, California, USA). Differences of p≤0.05 were considered significant.

### 2. Results

### 2.1 Schematic Representation of an Embodiment of the Method According to the Invention

### O₂ Plasma (Step 1 in FIGS. 1A-1D)

Hydroxyl groups on the surface of materials can react with chemical groups of other molecules and thus form a covalent bond.

3×3 cm polymethylpentene hollow fiber membranes (PMP, OXYPLUS, 3M Membrana, Wuppertal, Germany) were first treated with oxygen plasma to generate hydroxyl groups (—OH) on the surface. This was carried out using the Denta Plas® system from Diener electronic, Ebhausen, Germany. For this purpose, the membranes were treated with oxygen plasma at a pressure of 0.3 mbar (+0.20 mbar) and a plasma strength of 80% (+5%) in a low vacuum. This was followed by silanization.

Silanization with 3-Aminopropyltriethoxysilane (APTES) (Step 2 in FIGS. 1A-1D)

In this step, the free hydroxyl groups react with APTES. The dilution of APTES in anhydrous toluene is a widely used method for silanization. For silanization, the membranes were incubated for 30 min in 2% APTES (APTES, Sigma-Aldrich, Darmstadt, Germany) diluted in toluene (Sigma-Aldrich, Darmstadt, Germany) at 20 rpm and RT. Subsequently, three washing steps were performed for 2 min in an ultrasonic bath to remove unbound or weakly bound APTES. First, the membranes were washed in fresh 100% toluene solution followed by 50% toluene in 50% methanol and finally washed in 100% methanol and dried.

### DBCO Conjugation (Step 3 in FIGS. 1A-1D)

The silanized membranes were then incubated with 400 M DBCO-PEG₄-NHS ester (Jena Bioscience, Jena, Germany) diluted in Dulbecco's phosphate-buffered saline (DPBS, Invitrogen) for 30 min at RT. After incubation, three washing steps were carried out in DPBS for 5 min each. The DBCO used contains a PEG4 chain to extend the DBCO in order to facilitate binding to the metabolically modified cells. Furthermore, the DBCO used contains an activated carboxylic acid (—NHS ester), which can react with amino groups from APTES and thus form covalent bonds.

### 2.2 Detection of the Functional Groups Formed on the Surface of PMP-HFMs

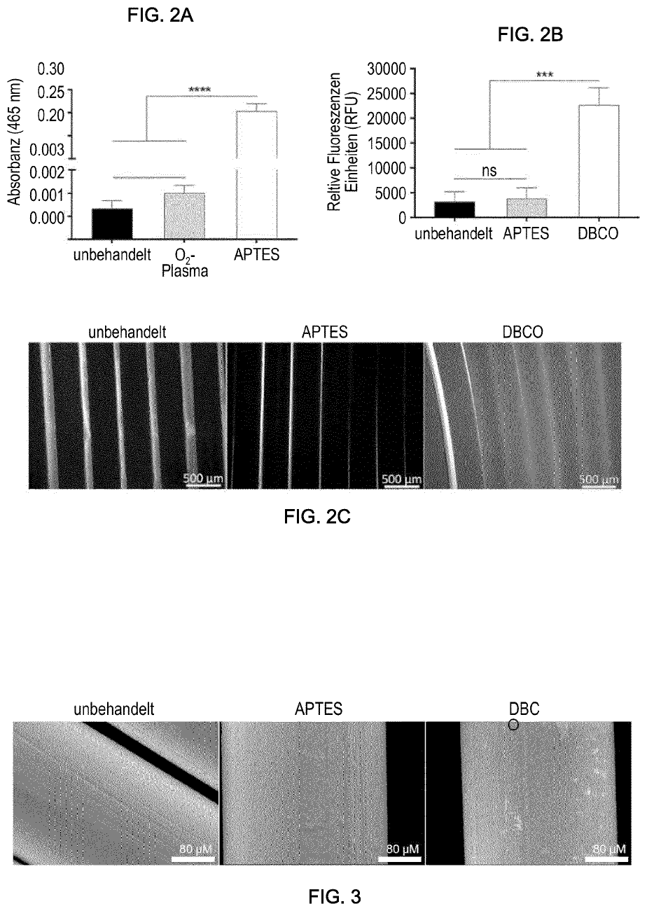

To detect the formed amino groups on untreated, O₂ plasma- and APTES-treated HFM membranes, the membranes were stained with methyl orange (FIG. 2A). APTES-treated membranes showed approximately 126-fold higher methyl orange binding (0.2523±0.0175 versus 0.002±0.0175) compared to O₂ plasma-treated membranes, indicating successful silanization of the surface and the presence of amino groups. These amino-functionalized surfaces were treated with DBCO-PEG₄-NHS ester and the presence of DBCO was detected by the click reaction of Cy3 azide on the surface. The fluorescence intensity of the HFMs was determined using a fluorescence reader (FIG. 2B). Surfaces treated with DBCO-PEG₄-NHS ester showed approximately 5.87-fold higher Cy3-azide conjugation than HFMs treated with APTES (RFU: 22553±3564 versus 3845±2155). Moreover, Cy3 fluorescence staining was only detected on the surface of DBCO-PEG₄-NHS ester-treated HFM by fluorescence microscopy analysis (FIG. 2C). Moreover, the SEM analyses showed no visual differences on the surface of the HFMs treated with APTES only or APTES and DBCO-PEG₄-NHS ester compared to the untreated HFMs, indicating that the coating process has no negative effect on the integrity of the HFMs (FIG. 3).

### 2.3 Analysis of the Membrane Tightness

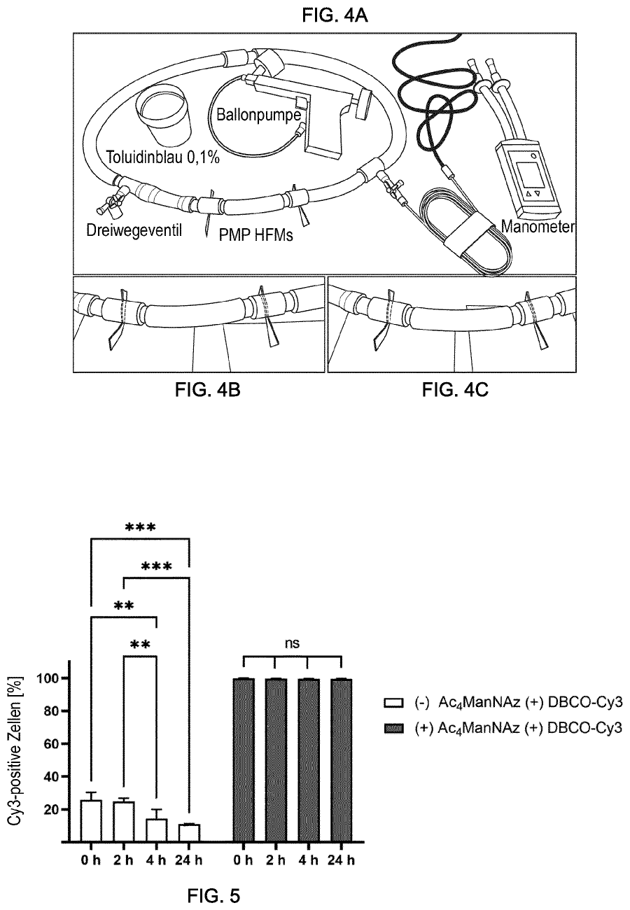

To investigate the effect of the functionalization process on membrane tightness, untreated and treated HFMs were placed in ECC-noDOP PVC tubes (⅜″× 3/32″, Raumedic AG, Helmbrecht, Germany) and fixed with an epoxy resin (UHU, Bühl/Baden, Germany) for 1 h at RT. The tubes were then filled with water (Fresenius Kabi, Bad Homburg, Germany) containing 0.1% toluidine blue (Sigma-Aldrich, Darmstadt, Germany) and a constant pressure of 250 mmHg±10 mmHg was generated for 3 h by connecting three-way valves to the tubes and using a balloon pump (Medtronic, Meerbusch, Germany). The pressure was monitored using a manometer (RS 1113, RS Components, Corby, United Kingdom) connected to the system. The tightness of the membranes was analyzed by placing a filter paper (0.83 mm thick, ThermoFisher, Waltham, USA) under the membranes (FIGS. 4A-4C). The images were taken 3 hours after incubation (n=3).

2.4 Analysis of the Presence of N₃ Groups after Removal of Ac4ManNAz from the Cell Culture Medium

To investigate the presence of N₃ groups after removal of Ac4ManNAz, 2×10⁵ HUVECs were seeded per well of a 6-well plate and treated with 50 M Ac4ManNAz or without in 2 ml medium for 48 h at 37° ° C. with 5% CO₂. Cells were washed with DBPS w Ca²⁺/Mg²⁺ and incubated in cell culture medium without Ac4ManNAz. After 0, 2, 4 and 24 h, the presence of N₃ on the cell surface was detected. For this purpose, the cells were washed with DPBS without Ca²⁺/Mg²⁺ and incubated with 5 μM DBCO-Sulfo-Cy3 (Jena Bioscience, Germany) in DPBS with Ca²⁺/Mg²⁺ for 1 h at 37° C. and 5% CO₂. Cells were then washed three times with 1 ml DPBS, detached with trypsin/EDTA (0.04%/0.03%) and centrifuged at 300×g for 5 min. The cell pellet was resuspended in 0.5 ml DPBS, and Cy3 labeling of 10 000 cells was analyzed by flow cytometry (FACS SCAN, BD, Heidelberg, Germany).

2.5 Metabolic Labeling of the Surface of Endothelial Cells with N₃ and Analysis of the Influence on Cell Viability

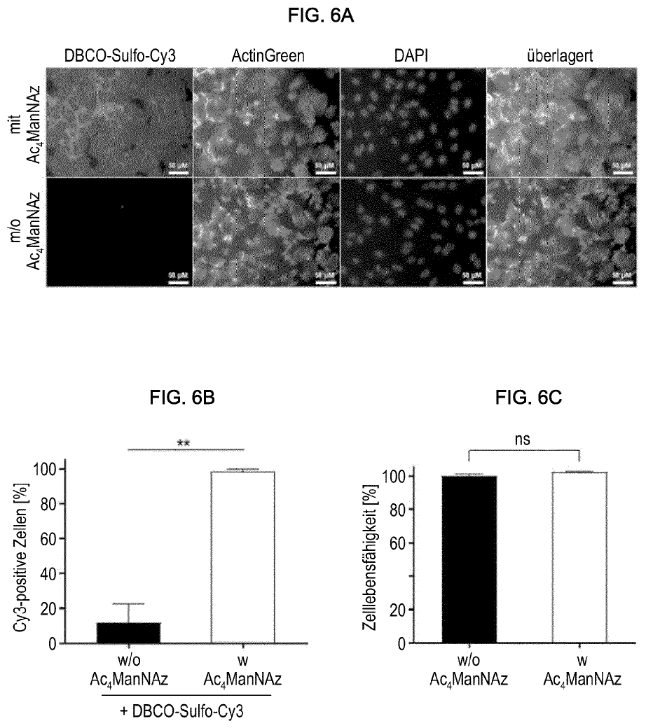

HUVECs were treated with 50 UM Ac4ManNAz for 48 hours. To determine whether N₃ groups were present on the cell surface, the cells were then incubated with 5 μM DBCO-Sulfo-Cy3 for 1 hour. The presence of N₃ on the cell surface led to conjugation of DBCO and labeling of the cells with Cy3 (FIG. 6A). The cells without Ac4ManNAz treatment were not stained. Furthermore, flow cytometric analysis showed that about 98% of the analyzed cells were Cy3-positive (FIG. 6B). Treatment of the cells with Ac4ManNAz had no effect on the viability of the HUVECs (FIG. 6C).

2.6 Endothelialization of Modified HFMs by Copper-Free Click Reaction of N₃-Labeled HUVECs on the DBCO-functionalized HFM surface

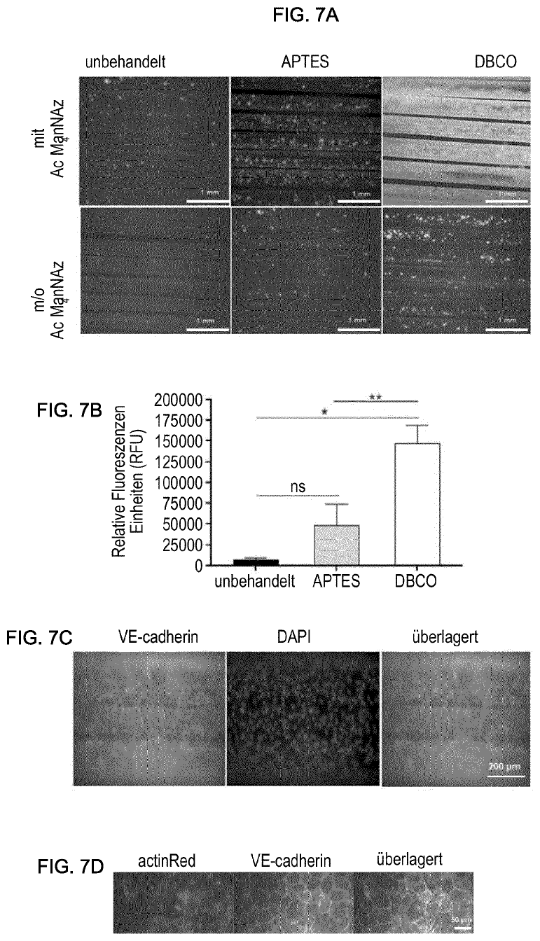

DBCO-coated and uncoated HFMs were incubated with 5.2×10⁶ Ac4ManNAz-labeled HUVECs for 24 hours under rotation and then for 24 hours under static conditions. The attached HUVECs were detected by staining with calcein AM (FIG. 7A). Fluorescence microscopy analysis showed that the Ac4ManNAz-treated HUVECs efficiently bound to the DBCO-functionalized HFM surface, leading to almost complete endothelialization. Detection of the fluorescence intensity of the HFMs also showed that DBCO functionalization led to significantly increased adhesion of HUVECs compared to uncoated or APTES-coated HFMs (FIG. 7B). The close cell-cell contacts of HUVECs on the HFM membranes were detected by staining with an anti-VE-cadherin antibody (FIG. 7C).

### 2.7 Reactivity of the Endothelial Layer to an Inflammatory Stimulus

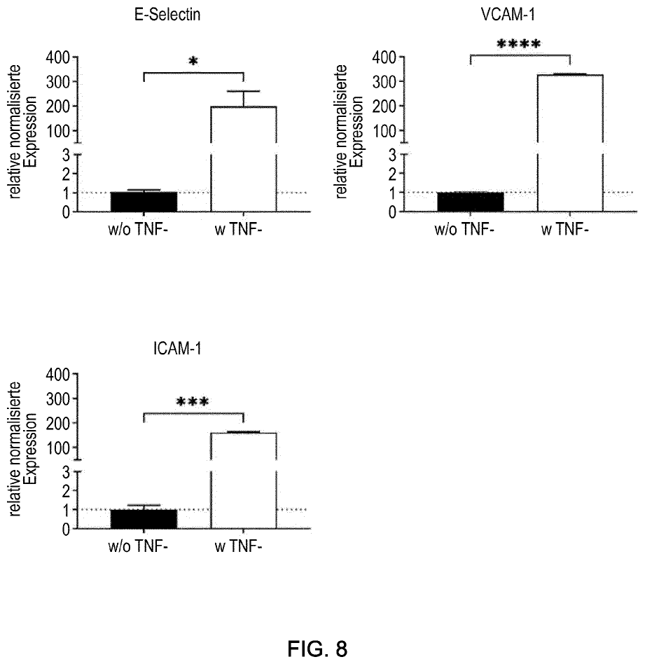

The expression of the adhesion molecules E-selectin, VCAM-1 and ICAM-1 was analyzed after stimulation of the endothelial layer on HFMs with TNF-α and compared with the non-stimulated endothelial layer by qRT-PCR (FIG. 8). Stimulation of the endothelial layer with TNF-α led to a significantly higher expression of E-selectin (199-fold), VCAM-1 (328-fold) and ICAM-1 (162-fold) compared to the non-stimulated endothelial layer. This demonstrated the responsiveness of the generated endothelial layer to HFMs.

### 2.8 Haemocompatibility of DBCO-Coated or Endothelialized HFMs

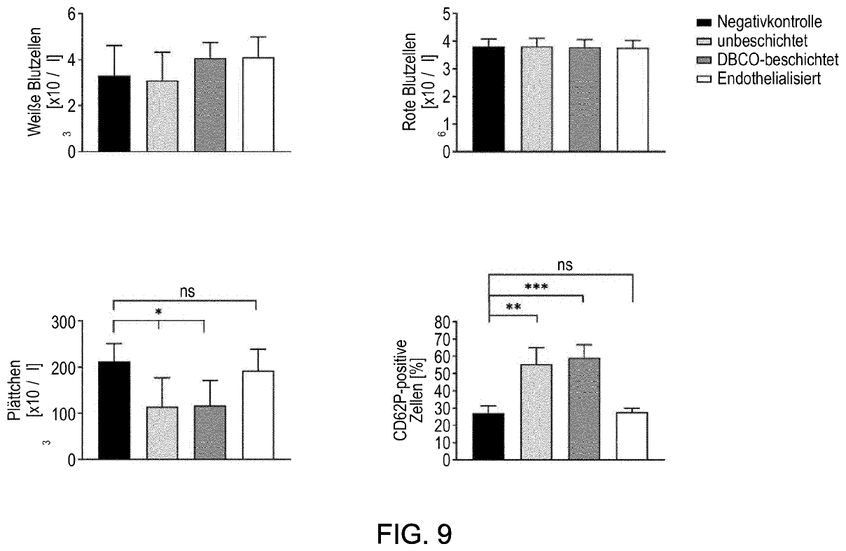

The influence of DBCO-functionalized or endothelialized HFMs on hemocompatibility was analyzed by dynamic incubation with fresh human blood. Blood cell count, platelet activation, coagulation, complement system and inflammation were determined after incubation of blood without HFMs (negative control), with uncoated, DBCO-coated or DBCO-coated and endothelialized HFMs. No significant differences in white or red blood cell counts were observed compared to blood samples without HFMs (FIG. 9). However, a significant decrease in platelet count was observed after incubation of blood with uncoated or DBCO-coated HFMs, while endothelialized membranes prevented the loss of platelets. In addition to platelet count, P-selectin expression on platelets was also analyzed as a marker for platelet activation. As shown in FIG. 9, a significantly higher number of activated platelets was detected on uncoated and DBCO-coated HFM compared to the blood samples without HFM. However, the endothelialized membranes prevented platelet activation.

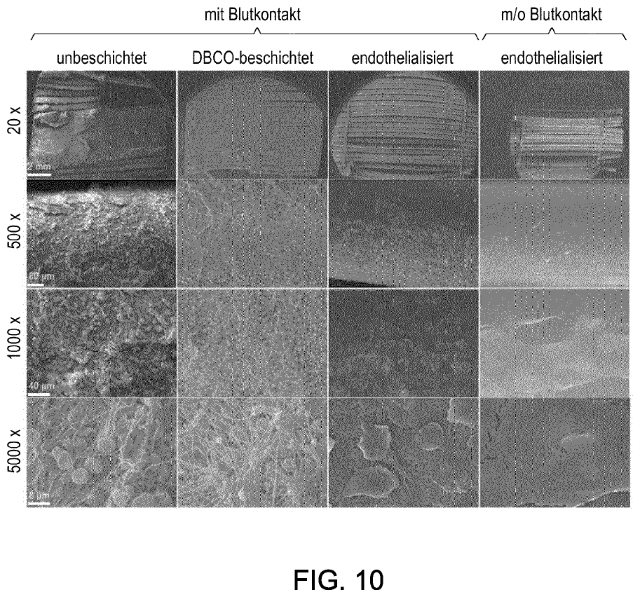

In addition, after incubation of the HFMs with blood, the adhesion of platelets and monocytes as well as the formation of fibrin on the surface of the HFMs was analyzed by SEM (FIG. 10). The endothelialization of the HFM surface led to the formation of a smooth, confluent endothelial cell layer. After incubation of unmodified HFMs with fresh human blood, a strong fibrin network with entrapped erythrocytes was observed on the surface. The DBCO-coated HFM surfaces also showed fibrin network formation. However, incubation of the endothelialized surface with human blood strongly prevented the formation of fibrin networks on the surface of the HFMs. In some places, cell-like structures were also observed, which could be exfoliated endothelial cells resulting from drying at the critical point or adherent monocytes.

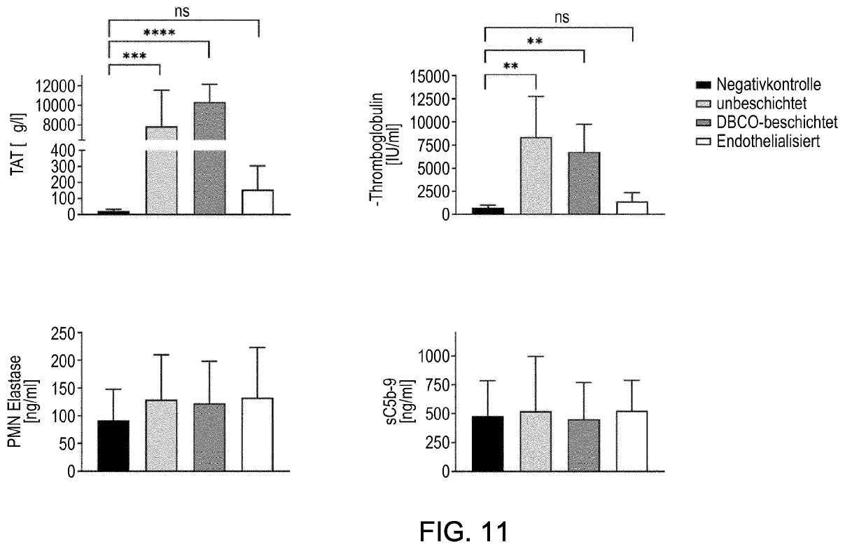

After incubation of blood with uncoated and DBCO-functionalized HFM, significantly increased levels of thrombin-antithrombin III complex (TAT), an indicator of coagulation activation, and ß-thromboglobulin, a marker of platelet activation, were detected (FIG. 11). In contrast, endothelialized HFM effectively prevented coagulation and platelet activation. With regard to the activation of inflammation (PMN elastase) and the complement system (sC5b9), no significant changes were measured between blood samples without HFM (negative control) and different HFM modifications.

### 2.9 Inflammatory Status of the Endothelium on HFMs

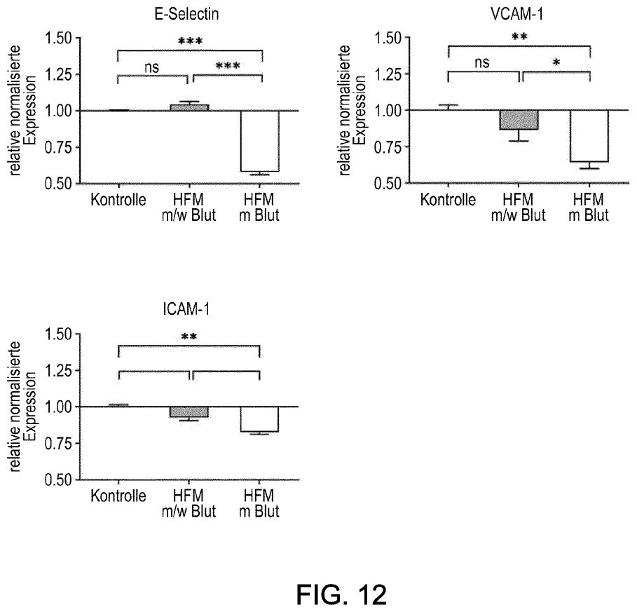

After endothelialization of HFMs, the expression of adhesion molecules was analyzed before and after incubation with blood for 90 minutes to determine the activation status of endothelial cells. HUVECs cultured in a 6-well plate served as a control. Interestingly, incubation of entothelialized HFM with blood reduced the expression of E-selectin and VCAM-1 compared to entothelialized HFM without blood contact (FIG. 12).

### 3. Conclusion

The inventors provide a method with which biological cells can be covalently bound to surfaces in a very specific and selective manner. This enables efficient colonization of the surfaces, e.g. with endothelial cells, in order to prevent the surfaces from being recognized as “foreign”. The method can also be used to bind the patient's own EPCs or “iPSC-derived” endothelial cells to desired surfaces of blood-contacting materials. Off-the-shelf products can be produced, which can be colonized with the patient's own endothelial cells if required. The conjugation of the cells to the surface takes place without a catalyst under physiological conditions.

Therefore, what is claimed, is:

1. A method for preparing a surface which can be colonized and/or is colonized with biological cells, comprising the following steps:
1. providing a surface comprising hydroxyl groups (—OH),
2. silanization of the surface containing hydroxyl groups,
3. conjugating a reactant reacting with azides in a copper-free click reaction to the silanized surface.

2. The method according to claim 1, comprising the following further step:
4. incubating the surface with biological cells which comprise azide groups (—N₃) on their surface under conditions which permit conjugation of the azide group with the reactant in a copper-free click reaction.

3. The method according to claim 1, wherein the reactant of step 3 is selected from the group consisting of: cycloalkyne, dibenzocyclooctyne (DBCO), cycloalkyne ester, DBCO-PEG₄-NHS ester, phoshin and phosphine ester.

4. The method according to claim 1, wherein in step 1 the provision of the surface containing hydroxyl groups is carried out by oxygen plasma treatment of a surface.

5. The method according to claim 1, wherein the surface is a surface of a medical device.

6. The method according to claim 5, wherein the medical device is selected from the group consisting of: artificial lung, oxygenator, artificial kidney, prosthesis, vascular prosthesis, stent, artificial heart.

7. The method according to claim 1, wherein the surface is a surface of a hollow fiber membrane (HFM).

8. The method according to claim 7, wherein the HFM is a polymethylpentene or polypropylene HFM.

9. The method according to claim 1, wherein the biological cells are endothelial cells.

10. A device having a surface which can be colonized with biological cells, wherein the surface comprises a reactant which is configured to react with azides in a copper-free click reaction.

11. The device according to claim 10, wherein the surface which can be colonized with biological cells was obtained by the method according to claim 1.

12. The device according to claim 11, wherein the device is selected from the group consisting of artificial lung, oxygenator, artificial kidney, prosthesis, vascular prosthesis, stent, artificial heart.

13. The device according to claim 11, wherein the device is a hollow fibre membrane (HFM).

14. The device according to claim 13, wherein the HFM is a polymethylpentene or polypropylene HFM.

15. A method for colonizing a surface with biological cells, comprising the following steps:
1. providing a surface comprising a reactant reacting with azides in a copper-free click reaction, and
2. incubating the surface with biological cells which have azide groups (—N₃) on their surface under conditions which permit conjugation of the azide group with the reactant in a copper-free click reaction.

16. The method according to claim 15, wherein the surface comprising a reactant reacting with azides in a copper-free click reaction was obtained by the process according to claim 1.

17. The method according to claim 15, wherein the biological cells having azide groups (—N₃) on their surface were obtained by incubation of biological cells with an azide sugar.

18. The method of claim 17, wherein the azide sugar is selected from the group consisting of: Ac4ManNAz (N-azidoacetylmannosamine-tetraacylated), N-azidoacetylglucosamine-tetraacylated (Ac4GIcNAz), N-azidoacetylgalactosamine-tetraacylated (Ac4GaINAz) and other azide-functionalized glycoconjugates.

19. The method according to claim 15, wherein the biological cells are endothelial cells.
